(19) United States
(12) Patent Application Publication
(10) Pub. No.: US 2013/0046347 A1
(43) Pub. Date: Feb. 21, 2013

(54) BENDABLE BONE PLATE FOR EXTERNAL FIXATION

(71) Applicants: Cheng-Kung CHENG, Taipei City (TW); Ching-Hou MA, Kaohsiung City (TW); Yu-Shu LAI, Taipei City (TW)

(72) Inventors: Cheng-Kung CHENG, Taipei City (TW); Ching-Hou MA, Kaohsiung City (TW); Yu-Shu LAI, Taipei City (TW)

(21) Appl. No.: 13/274,078

(22) Filed: Oct. 14, 2011

(30) Foreign Application Priority Data
Aug. 18, 2011 (TW) 100215400

(51) Int. Cl. A61B 17/80

(52) U.S. Cl. 606/283

(57) ABSTRACT

The present invention relates to a bendable bone plate for external fixation, comprising a main body made of flexible materials, wherein the end of the main body has a larger surface area. There are several perforations running the length of the bone plate body and more located on the larger proximal end. The characteristics of the present invention, a bendable bone plate for external fixation, deal with remote end fracture, proximal end fracture, central fracture or comminuted fracture of an upper limb long bone. Furthermore, the bendable bone plate for external fixation can be manipulated for averting the nerve plexus and traumatic wounds, allowing for external fixation surgery.

### BACKGROUND

1. Field of the Invention

The present invention relates to a fixation device for fracture of human bone structure. More specifically, it relates to a bendable bone plate for external fixation with the characteristics of a bendable bone plate that can avert the nerve plexus and traumatic injury and allow for external fixation surgery.

2. Description of the Related Art

Depending on the location of the fracture, fracture of long bones can be divided in to three categories; proximal end fracture, remote end fracture and central fracture. Furthermore, depending on the severity of the fracture, the proximal fracture can be categorized as 1-part to 4-part. Backbone fracture can also be categorized as simple or complex according to the comminution, or even as an open fracture caused by severe trauma. A bone plate design for proximal fractures can be found in U.S. Patent No. US2008/0119895, wherein a bushing is added to the screw hole of the bone plate and the traditional one way screw is replaced by a multi-axial screw. A design for remote fractures can be found in U.S. Patent No. US2007/0043368. The common point of the above-mentioned patents is that the fractures are secured using internal fixation. Studies have shown that, in the case of proximal upper limb long bone fracture, external fixation can maintain the tubular shape of the fracture, provide mobility through the early stages and allow better cicatrization and functional recovery when compared to internal fixation treatment.

Additionally, dealing with open wounds is the toughest part of fracture treatment mainly because the surface wound has to be treated before mending the surrounding tissue and the treatment of the fracture can be processed only when the surface skin recovers to a certain degree. However, cicatrization of the bone is often poor. Therefore, this external fixation bone plate aims to solve some of the complications associated with upper limb long bone fractures through an open or minimally invasive surgical approach. In the case of severe open wound patients, bone fixation can proceed once the surface wound is treated, which reduces the burden on patients undergoing the surgery and allows for the recovery of upper limb function in the shortest time. Most of the treatments for upper limb long bone fractures use bone plate fixation. However, with the large amount of nerve plexus in the upper limb, the nerves are susceptible to injury during the surgery and may cause complications.

### SUMMARY

The present invention aims to solve the problems associated with internal fixation bone plates through the use of an external fixation device.

Furthermore, this novel external bone plate can be bent or twisted according to the position of the fracture and, during the fracture surgery, avoids the wound in order to allow normal cicatrization of the wound and lowers the possibility of nerve injury.

The main body of the bone plate is made of flexible metal with one end of larger area corresponding to the shape of the proximal rotor head of the humerus or a remote joint surface of the humerus. The flexible metal maybe for example, but is not limited to, stainless steel, titanium, etc. The larger end of the bone plate can be used to cover the proximal or the remote part of the long bone in order to fasten the long bone at the proximal or remote position. There are several perforations on the main body that allow the bone pegs to pass through the bone plate, enter the bones via the specific perforations and fix the upper limb long bone by external fixation. The bendable bone plate for external fixation can be used for external fixation of the radius, humerus or ulna.

### DETAILED DESCRIPTION

The attached figures are offered only as an example of possible locations where the novel external fixation bone plate may be applicable. The bone plate is not limited to the locations illustrated.

The following is the description and the utilization method of the preferred embodiment of the present invention, the bendable bone plate, for external fixation.

As shown in FIG. 1, the basic structure of the bendable bone plate consists of a bone plate main body (1), a bulgy end (12) situated at the front of the bone plate, and several perforations (13) that allow bone pegs (not shown) to pass and enter the upper limb long bone.

FIG. 2 illustrates a preferred usage of the bendable bone plate whereby it is used as an external fixator for a fracture of the proximal humerus. The fracture is located through medical imaging of the long bone and the larger end of the external bone plate (12) is positioned to cover the proximal rotor head (31). Once the plate is positioned accurately, bone pegs (2) are inserted through the perforations (13) in the bone plate and are secured into the proximal rotor head of the humerus (31). From here, the remaining bone pegs may be inserted and secured into the bone at desirable locations.

As shown in FIG. 3, which is for illustrating a preferred embodiment of the present invention of bendable bone plate for external being used in a fracture surgery of remote humerus fracture. The doctor gets the position of the fracture by means of medical images, uses the bendable bone plate for external fixation of the present invention, wherein the surface of the bulgy part (12) at the front of the bone plate is enough to cover the remote joint surface (32) of the humerus. The position of the remote joint surface (32) is then obtained by the medical images. The bone peg (not shown) is fixed to the remote joint surface (32) of the humerus through the perforation (13) at the front (12) of the bone plate. Further fixation is done from the remote end of the humerus (3) to the proximal end of the humerus (3). The bone peg (2) is fixed to humerus (3) through the perforation (13) on the bone plate (1) to process the external fixation surgery of remote end humerus fracture.

FIG. 4 illustrates a preferred usage of the bendable bone plate for avoiding contact with a traumatic wound during surgery to secure a fracture at the proximal end of the humerus.The fracture is located through medical imaging and it's proximity to the traumatic wound is evaluated. A bench vice may be used to bend the bone plate into a desirable configuration so as to not disrupt the open wound (4). The larger end of the bone plate (12) is positioned to cover the proximal rotor head (31). Once the plate is positioned accurately, bone pegs (2) are inserted through the perforations (13) in the bone plate and are secured into the proximal rotor head of the humerus (31). From here, the remaining bone pegs may be inserted and secured into the bone at desirable locations to avoid further disruption of the open wound.

While the aforementioned applications of this bendable external fixation bone plate encompass the preferred locations, it is not limited to these locations. Various modifications may be made to this invention without departing from the scope covered by the appended claims. Any modification or change not departing from the main idea of the present invention is within the scope of the patent claimed.

### BRIEF DESCRIPTION OF THE DRAWINGS

FIG. 1 Basic structure of the present invention, a bendable bone plate for external fixation.

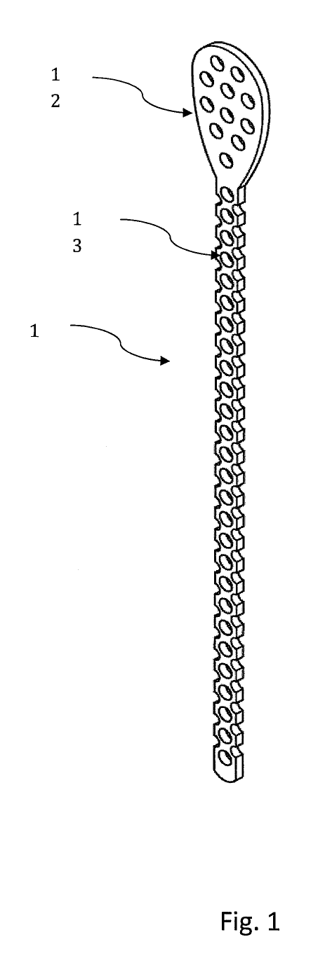

FIG. 2 (a) Front view of a preferred embodiment of the bendable bone plate used in a proximal end fracture surgery.

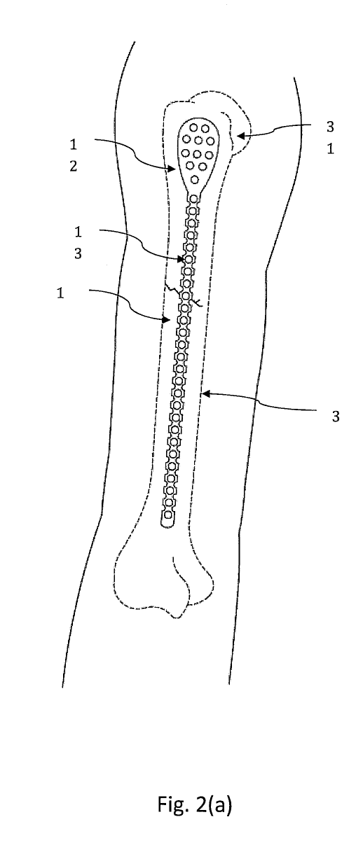

FIG. 2 (b) Side view of a preferred embodiment of the bendable bone plate used in a proximal end fracture surgery.

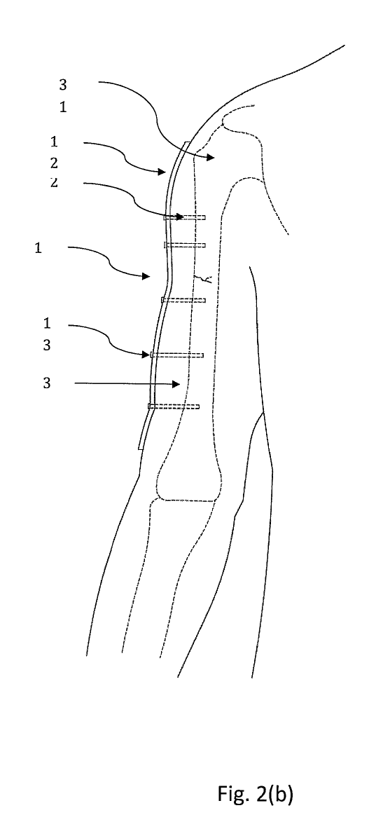

FIG. 3 Preferred embodiment of the bendable bone plate used in a remote end fracture surgery.

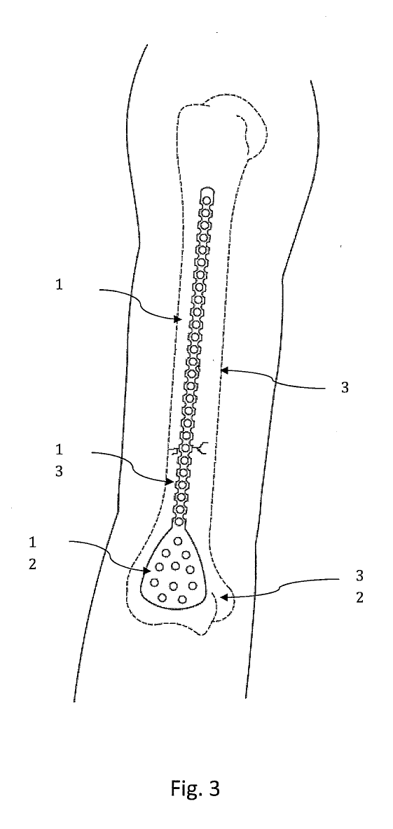

FIG. 4 Preferred embodiment of the bendable bone plate used in a proximal end fracture surgery.

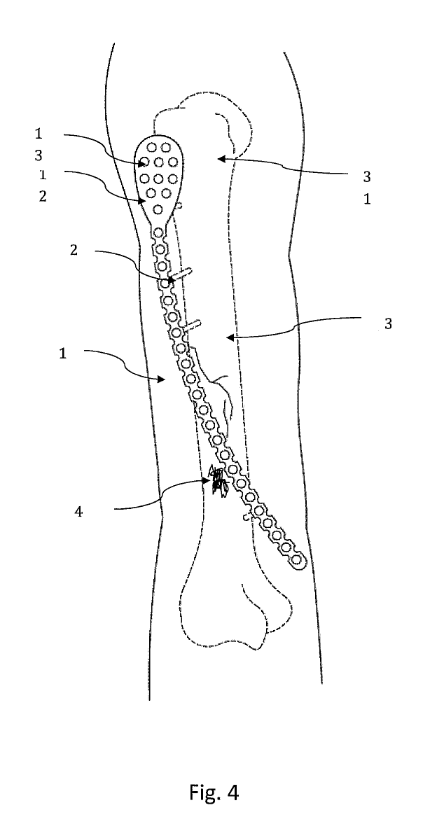

What is claimed is:

1. A bendable bone plate for external fixation that includes:
a main body of the bone plate which is made of flexible materials, wherein the proximal end of the bone plate has a larger surface area. There are several perforations running the length of the main body and several located on the larger end of the plate.

2. A bendable bone plate for external fixation as in claim 1, wherein the bone plate is used on upper limb long bones.

3. A bendable bone plate for external fixation as in claim 2, wherein the upper limb long bone can be the radius, humerus or ulna.

4. A bendable bone plate for external fixation as in claim 1, wherein the bulgy part at end of the main body of the bone plate corresponds to the shape of the proximal rotor head or distal end of the upper limb long bone.

5. A bendable bone plate for external fixation as in claim 1, wherein the flexible metal is made of stainless steel or titanium.
